SEQUENCE LISTING (11 sequences)

SEQ ID NO: 1 - protein, 9 aa, Unknown
  component of acquired enamel pellicle
DSSEEKFLR

SEQ ID NO: 2 - protein, 18 aa, Artificial
  fusion peptide
DSSEEKFLRDSSEEKFLR

SEQ ID NO: 3 - protein, 23 aa, Artificial
  fusion peptide
DSSEEKFLRRKFHEKHHSHRGYR

SEQ ID NO: 4 - protein, 28 aa, Artificial
  fusion peptide
DSSEEKFLRVPLSLRKFHEKHHSHRGYR

SEQ ID NO: 5 - protein, 28 aa, Artificial
  fusion peptide
DSSEEKFLRVPAGLRKFHEKHHSHRGYR

SEQ ID NO: 6 - protein, 43 aa, Unknown
  component of acquired enamel pellicle
DSSEEKFLRRIGRFGYGYGPYQPVPEQPLYPQPYQPQYQQYTF

SEQ ID NO: 7 - protein, 60 aa, Unknown
  component of acquired enamel pellicle
MKFLVFAFILALMVSMIGADSSEEKFLRRIGRFGYGYGPYQPVPEQPLYPQPYQPQYQQY

SEQ ID NO: 8 - protein, 38 aa, Homo sapiens
DSHEKRHHGYRRKFHEKHHSHREFPFYGDYGSNYLYDN

SEQ ID NO: 9 - protein, 32 aa, Homo sapiens
DSHAKRHHGYKRKFHEKHHSHRGYRSNYLYDN

SEQ ID NO: 10 - protein, 24 aa, Homo sapiens
DSHAKRHHGYKRKFHEKHHSHRGY

SEQ ID NO: 11 - protein, 14 aa, Artificial
  fragment of Histatin-5
RKFHEKHHSHRGYR

(19) United States
(12) Patent Application Publication
(10) Pub. No.: US 2017/0305984 A1
(43) Pub. Date: Oct. 26, 2017

(54) STATHERIN PEPTIDES

(71) Applicant: THE UNIVERSITY OF WESTERN ONTARIO, London (CA)

(72) Inventors: Walter Siqueira, London (CA); Rajesh Gupta, London (CA)

(21) Appl. No.: 15/513,504

(22) PCT Filed: Sep. 24, 2015
(86) PCT No.: PCT/CA2015/050947

§ 371 Date: Mar. 22, 2017

Related U.S. Application Data
(60) Provisional application No. 62/054,663, filed on Sep. 24, 2014.

(51) Int. Cl. C07K 14/47; A61K 38/17; C07K 17/04; A61K 9/10; C07K 7/08; C07K 7/06; C07K 19/00; A61K 38/00

(52) U.S. Cl. CPC C07K 14/4723; C07K 7/08; A61K 38/17; C07K 7/06; C07K 17/04; A61K 9/10; A61K 38/00; C07K 19/00; C07K 2319/00

(57) ABSTRACT

A novel statherin-based fusion peptide is provided. The fusion peptide comprises the statherin peptide, DSSEEKFLR, or a functionally equivalent variant thereof, fused to an acquired enamel pellicle protein or peptide. The statherin-based fusion peptide is useful to treat dental demineralization. Also provided is hydrogel-encapsulated enamel-protective protein or peptides such as statherin, a statherin-based fusion peptide or a histatin.

### FIELD OF THE INVENTION

The present invention generally relates to synthetic proteinase-resistant peptides useful to treat dental demineralization, including statherin-based fusion peptides, as well as delivery systems for proteins and peptides useful to treat dental demineralization.

### BACKGROUND OF THE INVENTION

According to World Health Organization (WHO), today oral health has become important indicator of overall health, well-being & quality life. It is one of the most important health management issues all over the world, but unfortunately highly neglected for various reasons including socioeconomic & lack of awareness. Poor oral health leads to the development of more serious and sometimes deteriorating health complications. This includes direct/indirect involvement of poor oral health in the precipitation of cardiovascular diseases, stroke, development of diabetes, liver problems, adverse respiratory complications, pregnancy complications & many more health problems with often extreme consequences

Dental caries is the most common chronic disease in the Canadian population. Millions of Canadians lose teeth, endure pain, and develop oral infections that contribute to systemic diseases such as cardiovascular disease, diabetes mellitus, adverse pregnancy outcomes and pulmonary infections. Canada's total bill for dental services was estimated to be $8.8 billion in 2004. In terms of direct costs, dental care in Canada is now the second most expensive disease category after cardiovascular diseases in the majority of the population encompassing different age groups. Human oral cavity is one of the best examples of a close knit multicultural community of different types of microorganisms. This consortia comprises different species of gram positive and gram negative bacteria, covering a whole multitude of cocci, bacilli, actinomycetes and other motile as well as non-motile forms, including different types of yeast, fungi & viruses. As per conservative estimates, based on in vitro cultivation, PCR amplification, 16 S rRNA molecular typing and pyro sequencing technology, there are greater than 1000 different types of microorganisms, that coexist both as planktonic form, in the saliva, as well as multi-layered, mixed species biofilms in highly specific collaborative partnership on dental and other surfaces in the mouth. These biofilms are formed by interactions with AEP through the processes of co-adhesion & co-aggregation, and by specific cell-cell interactions between genetically diverse microorganisms, for example, intercellular interactions between Capnocytophaga gingivalis & Actinomyces israeii or between Prevotella loescheii & Streptococcus sanguinis, respectively.

In the oral cavity, acquired enamel pellicle (AEP) is a kind of integument or thin film that acts as a protective covering of the tooth enamel surface. It is a complex biological, multi-tier heterogeneous mixture of specific salivary proteins, fragments, small peptides, intact native proteins, lipids, carbohydrates and food particles. It functions as an interface between enamel surface and the first layer of microorganism biofilm in the oral cavity. On one hand, it protects the tooth surface by resisting enamel demineralization, promoting re-mineralization, reducing enamel mechanical damage during mastication and modulating the early microbial colonizer composition on AEP. On the other hand, it acts as a docking platform for many opportunistic pathogenic microorganisms including Candida albican and Streptococcus mutans, causal organisms of oral candidiasis and dental caries, respectively. These pathogens, through co-adhesion and co-aggregation with other early and late colonizers form a multilayered biofilm on AEP. Biofilms are highly complex, metabolically interdependent as well as being an independent community of multispecies. They are formed by the complex inter and intra species interactions between AEP proteins and oral microbial communities. Primarily they are made up of water (˜96-97%), carbohydrates (1-2%) & proteins (<1%). They have a highly intricate network of channels and fluid filled intercellular spaces for facilitating nutrient, enzyme and metabolite exchange, intercellular communication, and scavenging of waste products and other solutes. These networks also lead to local accumulation and removal of waste products due to differences in colony density, resulting in different pH gradient microenvironments.

The biofilm community composition is strictly dictated and governed by the AEP protein composition, highly specific interactions between microorganism and the component proteins of AEP. The major salivary protein families associated with AEP include acidic proline rich proteins (aPRPs), basic PRP, α-amylase, MUC5B, agglutinin, cystatins, histatins and statherin, respectively. AEP formation itself is a multistage, dynamic, highly competitive and selective adsorption process of early pellicle proteins onto tooth enamel. It represents around 5% of roughly 2300 proteins present in saliva. For caries to occur, bacteria in the mouth must first adhere to and colonize on tooth structures to form a biofilm (commonly referred to as dental plaque). A major driving force governing the types and quantity of organisms colonizing on the tooth surface is exerted by the acquired enamel pellicle. The AEP forms a relatively insoluble structure on tooth surfaces, which acts as the interface between the mineral phase of teeth and dental plaque. AEP exhibits many desirable characteristics for the mineral homeostasis of teeth including: 1) partial protection against enamel demineralization, 2) promotion of enamel remineralization, 3) prevention of crystal growth on tooth surfaces, 4) reduction of frictional forces during mastication, and 5) affecting the attachment of the early microbial colonizers. The AEP composition is of great interest in the field of preventative dentistry since the pellicle serves as a solid support for the development of oral biofilm. Moreover these biofilms are extremely resistant to antimicrobial agents compared to planktonic microorganisms due to presence of extracellular polymeric substance (EPS) generated by microorganisms themselves, that acts as impervious & protective covering of biofilms. They are not only resistant to the action of most available antibiotics, but also resist the phagocytic action of human immune cells. These biofilms are very difficult to control and eradicate. Recent emergence of wide spread resistance in pathogenic microbes against natural host defense, as well as multi drug resistance (MDR) for many available antibiotics and other microbicidal drugs have created more serious oral and overall health-related threats globally.

Many studies have been devoted to uncovering the nature of the acquired enamel pellicle (AEP). The proteins within the AEP primarily originate from salivary glands, bacterial products, gingival crevicular fluid, or oral mucosa. However, AEP peptides are merely products of these proteins after bacterial cleavage and may retain or augment the functional properties of parent proteins. The AEP plays a crucial role in dental homeostasis by a) neutralizing acids produced by bacterial metabolism and b) acting as a selectively permeable membrane for remineralization. It also helps dictate the composition of early microbial colonizers, which ultimately form the microbial biofilm. It has been discovered that mature AEP proteome has more than 130 different native as well as phosphorylated proteins ranging in size from 150 kDa-5 kDa, of which about 50% are small peptides. Based on the possible role of these proteins in AEP development, they have been classified into 3 major groups; Ca²⁺ binding proteins, PO₄⁻ binding proteins & proteins interacting with other salivary proteins. AEP proteins have also been classified according to their putative biological functions, including inflammatory responses, immune defense, antimicrobial activity and remineralisation capacity. Many research possibilities concerning the more intricate aspects of saliva and the AEP, such as protein interactions, diagnostics, and synthetic analogs, remain to be thoroughly investigated.

One of the AEP principal proteins is statherin, which is strongly effective at inhibiting primary and secondary calcium phosphate precipitation, leading to supersaturated saliva that aids in remineralizing enamel surfaces. Statherin's functional peptide resides at the N-Terminal. Recently, a naturally occurring AEP peptide from this region was identified as a member of the acquired enamel pellicle. This peptide consists of 9 amino acids, DSpSpEEKFLR (where Sp is a phosphorylated serine). This peptide chain, referred to as DR9, has shown a significant effect (p<0.05) on hydroxyapatite growth inhibition in all studied concentrations when compared to other native statherin peptides.

In view of the foregoing, it would be desirable to identify proteins and/or peptide fragments for use in oral health maintenance.

### SUMMARY OF THE INVENTION

It has now been found that statherin-based fusion peptides are useful to treat dental demineralization, including conditions which require remineralization of enamel surfaces.

Accordingly, in one aspect of the invention, a novel statherin-based fusion peptide is provided comprising the statherin peptide, DSSEEKFLR, or a functionally equivalent peptide thereof, fused with an acquired enamel pellicle protein or peptide

In another aspect of the invention, a method of treating dental demineralization is provided. The method comprises the step of contacting enamel surfaces of teeth with a statherin-based fusion peptide for a sufficient period of time to provide treatment.

In a further aspect, a hydrogel encapsulated enamel-protective protein/peptide is provided.

These and other embodiments of the present invention are described by in the detailed description that follows by reference to the figures.

### BRIEF DESCRIPTION OF THE FIGURES

FIG. 1 illustrates the amino acid sequence of human statherin (A), histatin-1 (B), histatin-3 (C) and histatin-5 (D);

FIG. 2 graphically illustrates release behavior of bovine serum albumin encapsulated in chitosan nanoparticles when incubated in different pH conditions for 120 minutes;

FIG. 3 graphically illustrates that chitosan nanoparticle protects histatin 5 against proteolytic degradation;

FIG. 4 graphically illustrates the effect of chitosan nanoparticles or DR-9 peptide on calcium phosphate crystal growth inhibition; and

FIG. 5 graphically illustrates growth kinetics of Streptococcus mutans UA159 in the presence and absence of chitosan nanoparticle encapsulated (CSn) Histatin 5 (His5).

### DETAILED DESCRIPTION OF THE INVENTION

In one aspect, a novel statherin-based fusion peptide is provided comprising the statherin peptide, DSSEEKFLR (SEQ ID NO:1), or a functionally equivalent variant thereof, fused to a second acquired enamel pellicle protein or peptide. The statherin-based fusion peptide is useful to treat dental demineralization.

The present fusion peptide comprises the statherin peptide, DSSEEKFLR, or a functionally equivalent variant thereof. The term “functionally equivalent variant” as it relates to the statherin peptide, or other proteins and peptides disclosed herein (such as histatin proteins and peptides) includes naturally or non-naturally occurring variants thereof that essentially retain the biological activity of statherin peptide, e.g. to treat dental demineralization. Non-naturally occurring synthetic alterations may be made to the statherin peptide to yield functionally equivalent variants which may have more desirable characteristics for use in a therapeutic sense, for example, increased activity or stability. Functionally equivalent variants of the statherin peptide may, thus, include analogues, fragments and derivatives thereof.

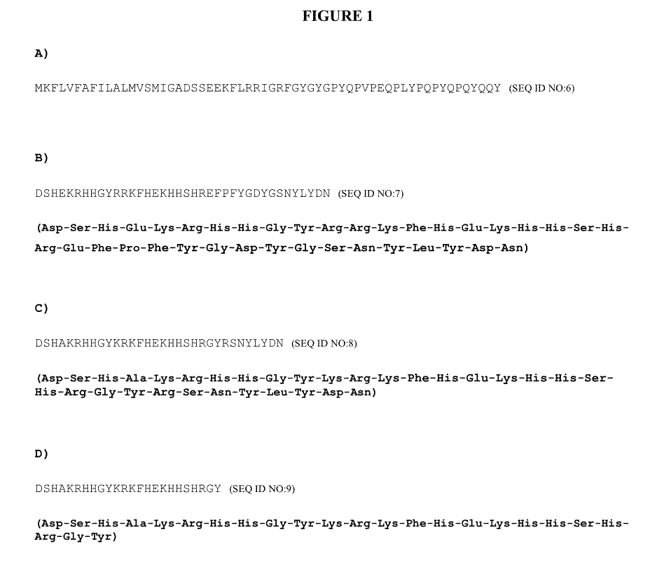

A functionally equivalent analogue of the statherin peptide in accordance with the present invention may incorporate one or more amino acid substitutions, additions or deletions. Amino acid additions or deletions include both terminal and internal additions or deletions to yield a functionally equivalent peptide. Examples of suitable amino acid additions or deletions include those incurred at positions within the protein that are not closely linked to activity. With respect to amino acid additions, in one embodiment, one or more amino acids that naturally exist within the statherin protein (as shown in FIG. 1) may be added to the statherin peptide, e.g. at either the N- or C-terminus of the peptide. Amino acid substitutions within the statherin peptide, particularly conservative amino acid substitutions, may also generate functionally equivalent analogues thereof. Examples of conservative substitutions include the substitution of a non-polar (hydrophobic) residue such as alanine, isoleucine, valine, leucine or methionine with another non-polar (hydrophobic) residue such as alanine, isoleucine, valine or methionine; the substitution of a polar (hydrophilic) residue with another polar residue such as between arginine and lysine, between glutamine and asparagine, between glutamine and glutamic acid, between asparagine and aspartic acid, and between glycine and serine; the substitution of a basic residue such as lysine, arginine or histidine with another basic residue; or the substitution of an acidic residue, such as aspartic acid or glutamic acid with another acidic residue.

A functionally equivalent derivative of the statherin peptide in accordance with the present invention is the statherin peptide, or an analogue or fragment thereof, in which one or more of the amino acid residues therein is chemically derivatized. The amino acids may be derivatized at the amino or carboxy groups, or alternatively, at the side “R” groups thereof. Derivatization of amino acids within the peptide may render a peptide having more desirable characteristics such as increased stability or activity. Such derivatized molecules include, for example, but are not limited to, those molecules in which free amino groups have been derivatized to form, for example, amine hydrochlorides, p-toluene sulfonyl groups, carbobenzoxy groups, t-butyloxycarbonyl groups, chloroacetyl groups or formyl groups. Free carboxyl groups may be derivatized to form, for example, salts, methyl and ethyl esters or other types of esters or hydrazides. Free hydroxyl groups may be derivatized to form, for example, O-acyl or O-alkyl derivatives. Also included as derivatives are those peptides which contain one or more naturally occurring amino acid derivatives of the twenty standard amino acids, for example: 5-hydroxylysine may be substituted for lysine; homoserine may be substituted for serine; and ornithine may be substituted for lysine. Phosphorylated derivatives are also encompassed, such as DSpSpEEKFLR. Terminal derivatization of the peptide to protect against chemical or enzymatic degradation is also encompassed including acetylation at the N-terminus.

The statherin peptide, and functionally equivalent variants thereof, may be made using standard, well-established solid-phase peptide synthesis methods (SPPS). Two methods of solid phase peptide synthesis include the BOC and FMOC methods. These peptides may also be made using any one of a number of suitable techniques based on recombinant technology. It will be appreciated that such techniques are well-established by those skilled in the art, and involve the expression of statherin peptide-encoding nucleic acid in a genetically engineered host cell. DNA encoding a statherin peptide may be synthesized de novo by automated techniques well-known in the art given that the protein and nucleic acid sequences are known.

A functionally equivalent variant need not exhibit identical activity to the statherin peptide, but will exhibit sufficient activity to render it useful to treat dental demineralization, e.g. at least about 25% of the biological activity of the statherin peptide, and preferably at least about 50% or greater of the biological activity of the statherin peptide.

To form the fusion peptide product according to the invention, the statherin peptide is fused to a second enamel-protective protein or peptide. The second enamel-protective protein/peptide may be an acquired enamel pellicle (AEP) peptide, or may be a synthetic enamel-protective peptide, or an enamel-protective peptide from another source, for example, a caesin phosphopeptide from yogurt extract or other milk source. In one embodiment, the second enamel-protective protein or peptide may be the statherin protein or peptide, i.e. to form a dimer, or may be a functionally equivalent peptide of the statherin peptide. In another embodiment, the second enamel-protective peptide may be a histatin, or a fragment of a histatin. For example, the second enamel-protective protein or peptide may be histatin-1, histatin-3 or histatin-5 (derived from proteolytic cleavage of histatin 3 at Tyr-24), a functionally equivalent fragment thereof, or a functionally equivalent natural or synthetic variant of any one of these. An example of a suitable fragment is a fragment of histatin-5, such as, RKFHEKHHSHRGYR (SEQ ID NO:10), referred to as RR14, or a functionally equivalent variant thereof as described above. In a further embodiment, the fusion peptide may include one or more additional copies of the statherin peptide or a different statherin peptide, or the second enamel-protective peptide or a different enamel-protective peptide.

The fusion peptide may be made using well-established techniques, as previously described. The fusion of the statherin peptide and the second enamel-protective peptide is conducted by a peptide linkage as opposed to a linking entity; however, a linker that does impact on the function of the fusion peptide may also be utilized.

Once prepared and suitably purified, the fusion peptide may be utilized in accordance with the invention to treat dental demineralization in a mammal. As used herein, the term “treat”, “treating” or “treatment” refers to methods that favorably alter dental demineralization, including those that moderate, reverse (i.e. remineralize), reduce the severity of, or protect against, the progression thereof, as well as methods useful to treat candidiasis, gingivitis, other oral disease, and the like. Dental demineralization refers to destruction of the hard tissues of the teeth (e.g. enamel, dentin and/or cementum), generally as a result of the harsh acidic environment of the oral cavity. Demineralization and other unfavourable conditions may also result from harmful microorganisms in the oral cavity, e.g. such as caused by S. mutans and C. albicans. Dental demineralization, thus, may result in tooth decay or dental caries and/or dental erosion. As used herein, the term “mammal” is meant to encompass, without limitation, humans, and domestic animals such as dogs, cats, horses, cattle, swine, sheep, goats and the like.

The fusion peptide may be administered either alone or in combination with at least one pharmaceutically acceptable adjuvant, in the treatment of dental demineralization in accordance with an embodiment of the invention. The expression “pharmaceutically acceptable” means acceptable for use in the pharmaceutical and veterinary arts, i.e. not being unacceptably toxic or otherwise unsuitable. Examples of pharmaceutically acceptable adjuvants are those used conventionally with peptide- or nucleic acid-based drugs, such as diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. Compositions for oral administration via tablet, capsule, solution or suspension are prepared using adjuvants including starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Creams, gels, pastes or ointments may be prepared for topical application using an appropriate base such as a triglyceride base, and may also contain a surface active agent. Aerosol formulations may also be prepared in which suitable propellant adjuvants are used. Other adjuvants may also be added to the composition regardless of how it is to be administered, for example, anti-microbial agents may be added to the composition to prevent microbial growth over prolonged storage periods.

To treat dental demineralization, a therapeutically effective amount of the fusion peptide is administered to a mammal. The term “therapeutically effective amount” is an amount of the statherin fusion peptide sufficient to provide a beneficial effect, while not exceeding an amount which may cause significant adverse effects. Dosages of the fusion peptide that are therapeutically effective will depend on many factors including the nature of the condition to be treated as well as the particular individual being treated. Appropriate dosages for use include dosages sufficient to exhibit statistically significant reduction in mineral loss in dental enamel. In one embodiment, dosages within the range of about 100 ng to 100 μg are appropriate.

In the present treatment, the fusion peptide may be administered by a route suitable to access the teeth, for example, oral or topical application. In one embodiment, the fusion peptide is provided in the form of a solution for use as a dental rinse to be swished around the teeth for a sufficient amount of time to treat dental demineralization. The fusion peptide may alternatively be provided as a solid, e.g. in the form of a tablet, capsule or powder, which may be prepared into a solution (by addition of a suitable liquid, such as water) for use as a rinse. In another embodiment, the fusion peptide is provided in the form of a gel or paste to be topically applied to the teeth, or to be used to brush the teeth. The fusion peptide may also be provided in a chewing gum and other chewable editable or non-edible items (e.g. teething products, animal chew toys, chewable bones and the like), film/gel strips or wafers, or coated on dental hygenic products such as tooth brushes, dental floss, dental picks and other devices used to clean teeth, as well as other orally used medical devices.

As one of skill in the art will appreciate, the fusion peptide may be administered to a mammal in the present method in conjunction with a second therapeutic agent to facilitate treatment of dental demineralization. The second therapeutic agent may be administered simultaneously with the fusion peptide, either in combination or separately. Alternatively, the second therapeutic agent may be administered prior or subsequent to the administration of the fusion peptide. Examples of such a second therapeutic agent include another agent useful to treat dental demineralization including, but not limited to, fluoride, a casein phosphopeptide, amorphous calcium phosphate, whitening agents such as peroxide or sodium bicarbonate, sealants, freshening and/or anti-microbial agents, herbal extracts of medicinal plants (e.g. Meswak™ (extract from Salvadora persica plant), Neem (Azadirachta indica plant), walnut and the like, in powder or fibrous form), and combinations thereof.

The fusion peptide may also be administered as a nucleic acid construct encoding the fusion peptide. Thus, a construct comprising nucleic acid sequence encoding a statherin peptide, such as DSSEEKFLR, the phosphorylated version thereof (DSpSpEEKFLR), or a functionally equivalent variant thereof, fused to a nucleic acid encoding a second acquired enamel pellicle protein or peptide, may used to treat dental demineralization. Such a construct may be administered to a mammal using any appropriate technique for administration of nucleic acid at a dosage sufficient to express a therapeutically effective amount of the fusion peptide, e.g. about 100 ng to 100 μg of fusion peptide.

In another aspect of the invention, an enamel-protective protein/peptide is provided in a biocompatible polymeric delivery system for administration to the oral cavity to treat dental de-mineralization. Suitable enamel-protective proteins or peptides includes those which protect the tooth surface by resisting enamel demineralization, promoting re-mineralization, reducing enamel mechanical damage during mastication and protecting against microbial colonization and damage. Examples of suitable enamel-protective proteins/peptides include, but are not limited to, the fusion peptide herein described, statherin or functionally equivalent peptides thereof, or histatin proteins/peptides (e.g. histatin-1, histatin-3, histatin-5 or functionally equivalent fragments thereof as described herein). For example, hydrogel delivery systems such as chitosan, alginate, collagen, poly(allylamine), functionally equivalent derivative hydrogels (e.g. modified versions of these hydrogels which maintain biocompatibility and encapsulation properties), or mixtures thereof, provide biocompatible particles that provide controlled release of encapsulated protein/peptide, thereby protecting the protein/peptide from degradation in the harsh environment of the oral cavity. Protein release is triggered by manipulating a physical or chemical stimuli, such as pH, ionic strength, temperature, magnetic field or biological molecules. For example, chitosan provides controlled release of encapsulated enamel-protective protein/peptide due to the pH sensitivity of chitosan. At pH values above 6.5, due to deprotonation of the chitosan matrix, the repulsion between chitosan polymers is reduced. This results in the shrinkage, tightening and closing of the chitosan pores preventing release of enamel-protective protein/peptides, while under the acidic conditions of the oral cavity (pH 3-5), due to protonation, the repulsion between chitosan polymers increases. This leads to chitosan matrix swelling and opening of the pores that permit the release of the encapsulated enamel-protective protein/peptide in a spatio-temporal fashion in response to specific environmental cues. Similarly, alginate provides controlled release of encapsulated protein/peptide based on the ionic strength of their environment, e.g. at normal ionic strengths (such as that of saliva), alginate capsules retain their contents, while an increase in ionic strength will induce the alginate capsules to release their contents.

Hydrogel encapsulated protein and or peptides are prepared by combining the selected hydrogel solution and protein/peptide with a conventionally used cross-linking anion (such as pyrophosphate (PPi) and tripolyphosphate (TPP)) at a suitable pH (5-6) to permit gelling to achieve nanoparticles, preferably having a diameter in the range of 5-20 nm, e.g. an average diameter of about 10 nm. The amount of protein or peptide loaded into the nanoparticles will vary with the protein/peptide, however, generally an amount in the nanogram range may be achieved.

Hydrogel encapsulated protein and or peptide nanoparticles may be formulated for use and used in a manner as described for the statherin-based fusion peptides to treat dental de-mineralization, e.g. preferably formulated for oral or topical application. Appropriate dosages for use include dosages sufficient to exhibit statistically significant reduction in mineral loss in dental enamel. In one embodiment, a suitable nanoparticle dosage is the dosage necessary to deliver an amount of enamel-protective protein/peptide in the range of about 100 ng to 100 μg.

The nanoparticles may additionally be used in conjunction with a second therapeutic agent, as above-described, either in combination, simultaneously, prior to or subsequent to, to enhance the effect thereof.

Embodiments of the present invention are described in the following specific example which is not to be construed as limiting.

### Example 1—Statherin-Based Fusion Peptide

### Materials and Methods

Enamel sample preparation was done as previously described (Siqueira et. al. 2010, J Dent Res. 2010; 89: 626-630, the relevant contents of which are incorporated herein by reference). Briefly, human permanent first molars without defects were cleaned, rinsed, and sectioned. After having the roots removed, the crowns were sliced sagittally into 4 sections (each with a 300 μm thickness) using a diamond saw, followed by grinding to a thickness of 150 μm using sandpaper. Each specimen was coated with a layer of light-cured dental adhesive (3M ESPE Scotchbond™ Universal) and nail varnish, excluding an untouched 2 mm window on the natural surface enamel.

Samples were randomly divided into 7 groups (N=12 per group), as shown in Table 1.

TABLE 1
Constructed peptides, derived from statherin and histatin, used in this study.

| Group | Sample | Peptide Sequence |
|---|---|---|
| 1 | DR9 | DSpSpEEKFLR (SEQ ID NO 1) |
| 2 | DR9-DR9 | DSpSpEEKFLRDSpSpEEKFLR (SEQ ID NO: 2) |
| 3 | DR9-RR14 | DSpSpEEKFLRRKFHEKHHSHRGYR (SEQ ID NO: 3) |
| 4 | DR9-VPLSL-RR14 (Bridge 1) | DSpSpEEKFLRVPLSLRKFHEKHHSHRGYR (SEQ ID NO: 4) |
| 5 | DR9-VPAGL-RR14 (Bridge 2) | DSpSpEEKFLRVPAGLRKFHEKHHSHRGYR (SEQ ID NO: 5) |
| 6 | Statherin | DSpSpEEKFLRRIGRFGYGYGPYQPVPEQPLYPQPYQPQYQQYTF |
| | | (SEQ ID NO: 6) |
| 7 | Distilled water | None |

Each specimen was submerged in 1 mg/mL construct peptide solution or distilled water (control group) and incubated for 2 hours at 37° C. After this period, the samples were then submerged in 1 mL of demineralization solution (0.05M acetic acid; 2.2 mM CaCl₂; 2.2 mM NaH₂PO₄; pH 4.5) at 37° C. for 12 days. Afterward, they were rinsed thoroughly with distilled water and dried with filter paper to remove any remaining acid residue. The remaining 1 mL of acidic solution was used to assess the calcium and phosphate concentration released from enamel during the demineralization process.

The calcium concentration of the solution was assessed using a quantitative colorimetric calcium determination assay (QuantiChrom™ Calcium Assay Kit, Bioassay Systems, Hayward, Calif., USA) with a UV-visible spectrophotometer to determine the optical density at a wavelength of 612 nm. The phosphate concentration was assessed using a colorimetric assay (PiColorLock™ Gold Detection System, Innova Biosciences, Cambridge, U.K.) and UV-visible spectrophotometer, to determine the optical density at a wavelength of 635 nm. All samples were analyzed in duplicate. Statistical analysis was performed with ANOVA and the Tukey Range Test.

### Results

The mean phosphate and calcium concentration values and standard deviations are shown in Table 2. Means that do not share a letter are significantly different.

TABLE 2
Means/standard deviations of calcium and
phosphate released from human enamel

| Peptide | Mean PO₄ Conc. (mM) | Mean Ca Conc. (mM) |
|---|---|---|
| DR9-VPAGL-RR14 | 3.917 ± 0.82^{A} | 4.755 ± 1.35^{A} |
| (SEQ ID NO: 5) | | |
| Water | 3.634 ± 0.62^{A} | 4.164 ± 0.89^{A} |
| DR9-VPLSL-RR14 | 3.159 ± 1.85^{A} | 4.106 ± 2.13^{A} |
| (SEQ ID NO: 4) | | |
| DR9-RR14 (SEQ | 2.282 ± 2.01^{B} | 3.132 ± 2.46^{B} |
| ID NO: 3) | | |
| DR9 (SEQ ID | 0.849 ± 1.80^{B, C} | 1.455 ± 2.13^{B, C} |
| NO: 1) | | |
| Statherin | 0.681 ± 1.11^{B, C} | 1.117 ± 1.29^{B, C} |
| (SEQ ID NO: 6) | | |
| DR9-DR9 | 0.514 ± 0.18^{C} | 0.581 ± 0.16^{C} |
| (SEQ ID NO: 2) | | |

NOTE:
Different letter superscripts indicate statistical difference, and same letter superscripts indicate no statistical difference, according to Tukey's test.

The same relative results were obtained for both phosphate and calcium. Functional domains linked with bridges (DR9-VPAGL-RR14 and DR9-VPLSL-RR14) did not provide any increased demineralization protection over the control. Samples coated with DR9-DR9 exhibited the lowest mineral loss, which reveals amplified enamel demineralization protection. Combinatory peptide (DR9-RR14) held an intermediate value among the groups, being significantly different from both the control and DR9-DR9.

### Conclusions

Statherin and histatin functional domains linked with a specific amino acid sequence (bridges) do not provide any functional improvement in mineral homeostasis. Combinatory peptide (DR9-RR14) is able to maintain the biological function of one of the precursor proteins, statherin. Enamel demineralization protection was amplified by DR9-DR9 when compared to single DR9 or statherin, proving that functional domain multiplication is a strong protein evolution pathway.

### Example 2—Chitosan Microparticles

Chitosan Microparticle Construction:

Chitosan at different concentrations (0.05, 0.1, 0.25, and 0.35%) were dissolved in 0.1-2% acetic acid. These chitosan solutions were dried in a spray dryer instrument to produce chitosan microparticles. Spray drying parameters included: inlet temperature (157-175° C.) and outlet temperature (97-105° C.) temperature, and the liquid feed flow rate (1.5-5 ml/min) was optimized for fabrication of homogenous and uniform sized particles. The size range, surface morphology and topography of the CP particles were characterized using a Zeta-sizer and scanning electron microscopy (SEM). Preliminary data illustrate the ability to construct chitosan particles. This experiment was carried out using 0.25% chitosan (w/v) in 0.1% (v/v) acetic acid solution with 158° C. inlet and 97° C. outlet temperature and a flow rate of 1.5 ml/min. The collected particles were used for SEM imaging, where a particle size range of 1.5-3.0 μm was observed.

Chitosan Nanoparticle Construction:

Chitosan nanoparticles were constructed using an ionic gelation method. Identical concentrations of chitosan, as described above, were dissolved in acetic acid (1.75×concentration of chitosan). Sodium tri-poly pentaphosphate (STPP) solution (v/v) was mixed with chitosan solutions to construct nanoparticles. Different ionic gelation parameters such as chitosan concentration, chitosan/STPP mass ratio, and chitosan solution pH effect were optimized for construction of homogenous, mono-dispersive, and uniform sized particles. Zeta-sizer, zeta potential and TEM will be employed for characterization of size range, surface charge, morphology and topography of the chitosan particles. A pilot experiment was carried out to construct chitosan nanoparticles through CS/STPP ionic gelation reaction. Chitosan 0.25% (w/v) was dissolved in acetic acid solutions (v/v) (1.75×concentration of chitosan) with 0-0.5% Tween-80 surfactant under constant stirring on a magnetic stirrer overnight. The pH of the solution was adjusted to 4.0-5.9 with 1-2 M NaOH. STPP solution (0.21-6.72 mg/ml) was added drop wise to the chitosan solution with continuous overnight mixing on a magnetic stirrer at 200-1200 rpm. Lyophilized chitosan particles were subjected to TEM. Hydrodynamic zeta sizing of the colloidal suspension was carried out using dynamic laser scattering (DLS). Zeta sized distribution, where nanoparticles ranged from 9.8 to 11.8 nm, within an average size of 10.5 nm, was achieved.

Additional experiments were carried out to construct chitosan nanoparticles through chitosan/TPP ionic gelation reaction with chitosan concentration ranging from 0.01% to 0.1% dissolved in HCl solution (8 mM; 7 μl 12 M HCl/10 ml chitosan solution v/v). Tri-polyphosphate (TPP) concentration ranged from 0.01 to 0.1%. Initial chitosan and TPP solution pH was set to 5.5. Stirring speed was set to 800 rpm. TPP final concentration ranged from 0.01-0.022%. The final pH of nano suspension ranged from 5.6-6.0. All these parameters were tested to determine chitosan nanoparticle sizes. Chitosan nanoparticles were characterized using dynamic laser light scattering and Zeta potential. The chitosan nanoparticle hydrodynamic diameter ranged were from 120 to 750 nm. In addition, the nanoparticles demonstrated a positive surface charge, and chitosan nanoparticles showed a zeta potential range of 26.2±1.8 to 11.6±2.8 mV.

Encapsulation of AEP within Chitosan Micro- and Nanoparticles:

Based on the optimized parameters for construction of chitosan micro- and nanoparticles, AEP was mixed directly with the chitosan solution. Chitosan micro- and nanoparticles were constructed, as described above, and the particles were characterized through Zeta sizing and SEM. Reverse Phase-High Performance Liquid Chromatography (RP-HPLC) and/or enzyme-linked immunosorbent assay (ELISA) was used to determine the degree of encapsulation by measuring the amount of free peptides, before and after the encapsulation procedure.

Chitosan encapsulation of histatin 5, DR-9, RR-14 and bovine serum albumin was carried out to determine the chitosan encapsulation rate. ELISA assay assessed the remaining unencapsulated protein or peptide. The results demonstrated a protein/peptide encapsulation efficiency ranging from 75% to 93%, depending of the peptide or protein encapsulated (Table 2).

TABLE 2

| Protein/Peptide | Encapsulation Efficiency |
|---|---|
| Histatin 5 | 82.9% |
| DR-9 | 75.5% |
| Histatin 5-tag | 82.0% |
| RR-14 | 93.1% |
| Bovine Serum Albumin | 88.3% |

DR-9 encapsulated chitosan nanoparticles showed a zeta potential of 25.53±1.40. Thus, chitosan encapsulation produces stable nanoparticles which will facilitate delivery and distribution of AEP protein/peptides into the oral cavity.

pH-Induced Release Mechanism:

Encapsulated peptide release studies were performed in buffers encompassing a wide range of acidic to basic pH values (3, 4, 4.5, 5, 5.5, 6.8, 7, 7.4 and 11). The pH values for this study were carefully selected to correlate with the most common oral environmental episodes such as dental erosion (pH 3), dental caries (pH 4.5-5.5), or physiological salivary pH (pH 6.8). The equivalent of 800 μM of peptide encapsulated in chitosan was incubated in 5 ml of different pH-buffers at 37° C. and RT with 35 rpm agitation. The amount of peptide released from chitosan was measured at time intervals (0, 5, 10, 30, 60, 120, 300 minutes). RP-HPLC was used to determine the released level of peptide at each time-point. In addition, changes in size of chitosan particles due to pH induced swelling and shrinkage behaviour was measured through Zeta sizing and SEM as described above.

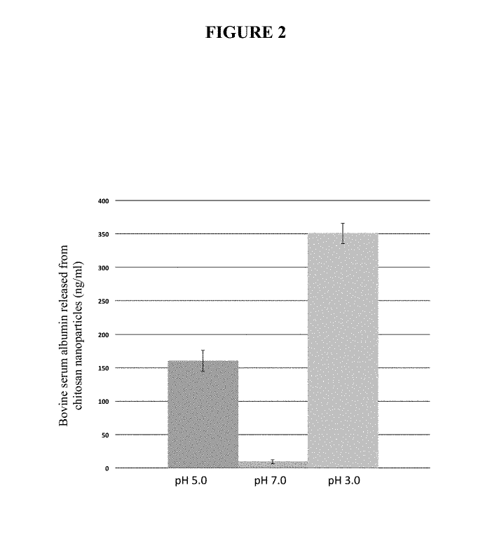

Nanoparticles of bovine serum albumin encapsulated with chitosan were incubated in buffers with pH 7.0 (phosphate buffer), pH 5.0 (acetate buffer) or pH 3.0 (acetic acid). ELISA assay was used to determine the protein release from chitosan nanoparticles after treatment with these specific pH conditions. At pH 3.0 (pH related to dental erosion), and pH 5.0 (pH related to dental caries) the release of bovine serum albumin was 35, and 17 times higher, respectively, than when subjected to pH 7.0 (pH related to the natural salivary pH) (FIG. 2).

Protection from Proteases:

The equivalent of 400 μM of salivary protein encapsulated with chitosan was added to 1:5 diluted whole saliva supernatant and further incubated at 37° C. for 0, 30, 60 and 120 min. In preliminary studies, the dilution of the saliva retards the degradation process and facilitates analysis of the salivary proteins. As controls, 400 μM of selected salivary protein without encapsulated chitosan was incubated with 1:5 diluted whole saliva supernatant for the same time-points. A second control was also employed, where salivary protein encapsulated with chitosan is incubated with distilled water at 37° C. for the same time-points. Immediately after the addition of whole saliva supernatant (t=0), and after each noted incubation period, samples were removed and then boiled for 5 minutes to abolish proteolytic activity. After being boiled, the samples were subjected to Reverse Phase-High Performance Liquid Chromatography (RP-HPLC) to finalize the survival/degradation level of salivary protein encapsulated (or not) with chitosan from each time-point. Briefly, samples were dried, re-suspended in 0.1% TFA, clarified with a 0.22-μm filter (Pall Corporation, Ann Arbor, Mich.), and applied to a C¹⁸ column (Vydac 218MS, 4.6×250 mm, Deerfield, Ill.) linked to an HPLC (Waters, Watford, UK). Tested salivary proteins and their potential protein fragments were eluted with a linear gradient from 0 to 55% buffer B containing 80% acetonitrile, 19.9% H₂O, and 0.1% TFA, over 110 min at 1.3 ml/min. The eluate from the RP-HPLC runs was monitored at 214 and 230 nm. The peak area related to the intact tested salivary protein was measured at all different time-points and transformed to the percentage value.

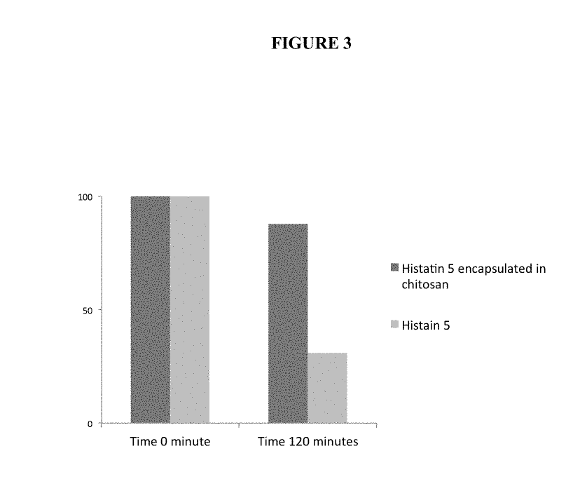

Histatin 5, both encapsulated in chitosan nanoparticles and non-encapsulated, were incubated in whole 1:5 diluted saliva supernatant. Results show that the chitosan nanoparticle was able to protect histatin 5 against proteolytic degradation, consequently increasing the lifetime of this protein in the whole saliva environment (FIG. 3).

Inhibition of Spontaneous Precipitation of Supersaturated Calcium Phosphate Solutions by Chitosan-Encapsulated AEP Proteins/Peptides:

To investigate the ability of the chitosan encapsulated AEP proteins/peptides to prevent crystal growth, microtiter plates were coated with proteins/peptides in a HEPES buffer at RT for 1 h. The wells were washed followed by the addition of a solution containing phosphate (15 mM; pH 7.4), NaCl (150 mM) and CaCl₂ (50 mM). The solutions were incubated for 4 h at room temperature, allowing the formation of hydroxyapatite crystals. After this period, the solution was removed, 5% Alizarin Red S (pH 4.2), was added, followed by cerylpyridinium chloride. Crystal production was analyzed spectrophotometrically at 570 nm.

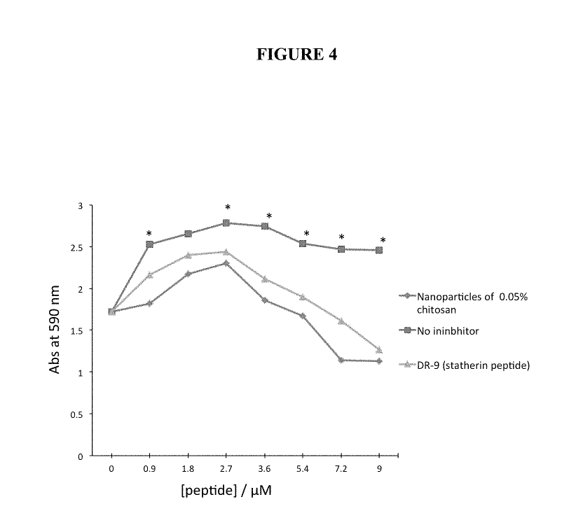

The results showed that both nanoparticles of chitosan and DR-9 (unencapsulated) under all test concentrations exhibited statistically significant inhibition of calcium phosphate crystal growth when compared to a group without any inhibitor (FIG. 4). This data demonstrated that DR-9, a small native AEP peptide, and chitosan nanoparticles have a beneficial effect in 1) preventing unwanted calcium phosphate crystal formation, 2) facilitating enamel remineralization, and 3) inhibition of dental calculus formation. Thus, encapsulation of specific AEP proteins/peptides, such as DR-9, with chitosan nanoparticles is expected to amplify the calcium phosphate crystal formation inhibitory effect.

Effects of Chitosan Encapsulated Salivary Proteins on Enamel Demineralization:

Demineralization studies are conducted using thin sections of human enamel. Enamel minerals were analyzed using microradiography. To determine the influence of selected chitosan encapsulated salivary proteins on enamel demineralization, resin-coated enamel sections were first exposed to solutions containing chitosan encapsulated salivary proteins at a concentration of 1.0 mg/ml for 2 h at 37° C. To mimic the acidic environment of dental caries, chitosan encapsulated salivary proteins and control sections were placed into individual tubes with 3 ml of a demineralization solution containing 2.2 mM CaCl₂, 2.2 mM NaH₂PO₄, 5 mM acetic acid, a pH 4.5. The sample was incubated at 37° C. with gentle agitation for a period of 12 days. All solutions contained 3 mM sodium azide as a bacteriostatic agent. Immediately after the demineralization period, the specimens were extensively washed with distilled water and dried with filter paper. Mineral loss was evaluated by comparing the microradiography taken before and after exposure to the acidic conditions.

Enamel pieces were prepared as described and coated with chitosan nanoparticles, DR-9 encapsulated in chitosan nanoparticles, DR-9, and 0.05% NaF (gold standard group). In some groups, parotid saliva was allowed to adsorb first on enamel species to mimic the AEP (Table 3). Adsorption was allowed to proceed for a period of 2 hours at 37° C. with gentle agitation. Enamel specimens were then washed with distilled water and immersed in a demineralization solution, pH 4.5 for 12 days. This solution was used to measure the amount of calcium and phosphate released from enamel. All coated groups showed a statistically significant higher protection than those not coated (control group). DR-9 group demonstrated an intermediary level of demineralization protection while DR-9 encapsulated with chitosan nanoparticles and NaF groups showed a better acid protection (Table 3).

TABLE 3

| | Calcium (mM) | Phosphate (mM) |
|---|---|---|
| Water (no DR-9 or chitosan) | 1.90 ± 0.17 ^{a} | 0.75 ± 0.21 ^{a} |
| 0.05% NaF adsorbed for 2 hrs | 0.30 ± 0.13 ^{b} | 0.17 ± 0.06 ^{b} |
| DR-9 chitosan nanoparticles | 0.47 ± 0.07 ^{c} | 0.22 ± 0.09 ^{c} |
| adsorbed for 2 hrs | | |
| Chitosan (blank nanoparticle) | 0.68 ± 0.10 ^{c} | 0.20 ± 0.03 ^{c} |
| adsorbed for 2 hrs | | |
| Parotid saliva adsorbed for 2 hrs | 0.32 ± 0.13 ^{b} | 0.14 ± 0.04 ^{b} |
| followed by DR-9 chitosan | | |
| nanoparticles adsorbed for 2 hrs | | |
| Parotid saliva adsorbed for 2 hrs | 0.44 ± 0.07 ^{c} | 0.19 ± 0.03 ^{c} |

Superscripts within each column denote no statistical difference according to Tukey's test among peptides and control.
p < 0.05.
n = 10 per group.

Effect of Chitosan-Encapsulated Histatin 5 on Growth of S. mutans:

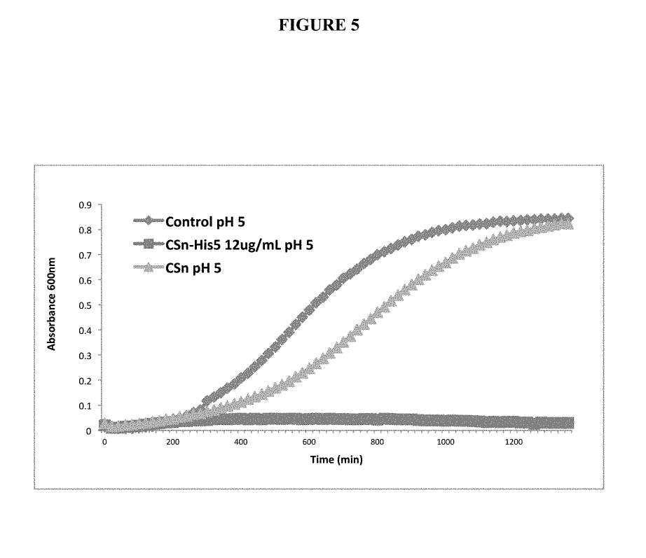

The effect of chitosan-encapsulated Histatin 5 (CSnp-His5) and controls on the growth of S. mutans UA159 strain was tested using a Chemically Modified Medium (CDM) at pH 5 (as described in Mashburn-Warren et al. Mol Microbial, 2010. 78(3): p. 589-606). Relative to the controls without chitosan supplementation, exposure of S. mutans early-lag phase cells to 12 μg/mL of chitosan encapsulated Histatin 5 constructs (CSnp-His5) led to complete growth inhibition (FIG. 5). On the other hand, exposure of S. mutans to empty chitosan nanoparticles (CSnp) led to impaired, but not abolished growth of S. mutans (FIG. 5).

Screening of Peptide Effects on Biofilm Formation of S. mutans:

S. mutans UA159 biofilms were formed on polystyrene microtiter plates for 18 h at 37° C. and 5% CO₂ in a Chemically Defined Medium (CDM) at pH 5 containing 10 μg/mL of chitosan-encapsulated histatin 5 (CSnp-His-5) nanoparticles. Controls biofilms were formed in CDM medium with unencapsulated chitosan vectors (CSnp) or in the presence of 1 mM potassium phosphate buffer with 0.5% Tween 80. Following incubation, supernatant was removed, biofilms dried and stained with 0.1% crystal violet solution.

Confocal Laser Scanning Microscopy (CLSM) was used to measure the growth of the biofilm. The results showed that biofilm formation was significantly impaired in the presence of 10 μg/mL of CSnp-His5 relative to the controls with or without CSnp. In addition, CSnp group without histatin 5 demonstrated a partial reduction in the biofilm growth, showing again the antimicrobial inhibitory effects of chitosan nanoparticles against bacteria under acidic environments. Thus, encapsulation of AEP proteins/peptides with chitosan enhances the biological activity of these compounds against S. mutans due to synergistic effects as a result of chitosan encapsulation and AEP peptide/protein antimicrobial activities.

1. A statherin-based fusion peptide comprising the statherin peptide, DSSEEKFLR, or a functionally equivalent variant thereof, fused to second enamel-protective protein or peptide.

2. The fusion peptide of claim 1, wherein the second enamel-protective protein or peptide is an acquired enamel pellicle (AEP) peptide.

3. The fusion peptide of claim 1, wherein the second enamel-protective protein or peptide is the statherin protein or a peptide fragment thereof.

4. The fusion peptide of claim 1, wherein the second enamel-protective protein or peptide is a histatin, or a functionally equivalent variant or fragment of a histatin.

5. The fusion peptide of claim 4, wherein the second enamel-protective protein or peptide is selected from the group consisting of histatin-1, histatin-3 and functionally equivalent fragments thereof.

6. The fusion peptide of claim 5, wherein the functionally equivalent fragment is selected from histatin-5 and RKFHEKHHSHRGYR.

7. The fusion peptide of claim 1, which is DSpSpEEKFLR-DSpSpEEKFLR.

8. The fusion peptide of claim 1, which is DSpSpEEKFLR-RKFHEKHHSHRGYR.

9. A composition comprising the statherin-based fusion peptide of claim 1 and a pharmaceutically acceptable carrier.

10. The composition of claim 9, formulated for oral administration.

11. The composition of claim 9, which is a solution, tablet, capsule, powder, gel or paste.

12. A method of treating dental demineralization comprising the step of contacting teeth with a statherin-based fusion peptide as defined in claim 1.

13. The method of claim 12, wherein the teeth are contacted with a dosage in the range of about 100 ng to 100 μg.

14. The method of claim 12, wherein the fusion peptide is used in conjunction with fluoride, a casein phosphopeptide, amorphous calcium phosphate, whitening agents, dental sealants, freshening agents, anti-microbial agents, herbal extracts from medicinal plants and combinations thereof.

15. An encapsulated enamel-protective protein or peptide which is encapsulated with a biocompatible hydrogel.

16. The encapsulated protein or peptide of claim 15, wherein the biocompatible hydrogel is selected from the group consisting of a hydrogel of chitosan, alginate, collagen, poly(allylamine), a functionally equivalent derivative of any of these and mixtures of these hydrogels.

17. The encapsulated protein or peptide of claim 15, wherein the protein or peptide is statherin or a functionally equivalent peptide thereof, a histatin protein or a functionally equivalent peptide thereof, or a fusion peptide as defined in claim 1.

18. The encapsulated protein or peptide of claim 16, wherein the hydrogel is a chitosan hydrogel.

19. The encapsulated protein or peptide of claim 17, wherein the peptide is selected from the group consisting of statherin, DSpSpEEKFLR, DSpSpEEKFLR-DSpSpEEKFLRD, SpSpEEKFLR-RKFHEKHHSHRGYR, histatin-1, histatin-3, histatin-5 and functionally equivalent histatin fragments.

20. A method of treating de-mineralization of teeth, comprising administering to the oral cavity a hydrogel encapsulated enamel-protective protein or peptide as defined in claim 15.
